(19) United States
(12) Patent Application Publication
(10) Pub. No.: US 2026/0176626 A1
(43) Pub. Date: Jun. 25, 2026

(54) Compositions And Methods For Transcription Factor 4 (TCF4) Repeat Expansion Excision

(71) Applicant: EmendoBio Inc., Wilmington, DE (US)

(72) Inventor: Rafi EMMANUEL, Ramla (IL)

(21) Appl. No.: 19/126,913

(22) PCT Filed: Nov. 2, 2023
(86) PCT No.: PCT/US2023/078551

§ 371 Date: May 2, 2025

Related U.S. Application Data
(60) Provisional application No. 63/382,108, filed on Nov. 2, 2022.
(60) Provisional application No. 63/482,870, filed on Feb. 2, 2023.

(51) Int. Cl. C12N 15/113; C12N 15/85

(52) U.S. Cl. CPC C12N 15/113; C12N 15/85

(57) ABSTRACT

RNA molecules comprising a guide sequence portion having 17-50 contiguous nucleotides containing nucleotides in the sequence set forth in any one of SEQ ID NOs: 1-2325 and compositions, methods, and uses thereof.

This application claims the benefit of U.S. Provisional Application No. 63/482,870, filed Feb. 2, 2023, and U.S. Provisional Application No. 63/382,108, filed Nov. 2, 2022, the contents of each of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “231102_92056-A-PCT_Sequence_Listing_AWG.xml”, which is 2,044 kilobytes in size, and which was created on Nov. 2, 2023 in the IBM-PC machine format, having an operating system compatibility with MS-Windows, which is contained in the XML file filed Nov. 2, 2023 as part of this application.

### BACKGROUND OF INVENTION

The Transcription factor 4 (TCF4) gene (also called ITF2 and SEF-2) is located on chromosome 18 and consists of 20 exons. Exons 1 and 20 are noncoding exons, with the other exons coding for a basic helix-loop-helix (bHLH) protein that functions as a homodimer or as a heterodimer with other bHLH proteins. These dimers bind DNA at Ephrussi (E) box sequences.

TCF4 has been reported to be expressed in the developing corneal endothelium. It plays a pivotal effector role in the Wnt signaling pathway. Expression of TCF4 is a key factor in controlling the balance between proliferation and differentiation. Wnt/TCF4 signaling has been reported to regulate human corneal endothelial cells (hCECs).

An intronic trinucleotide CTG repeat expansion of TCF4 may account for 50-70% of cases of Fuchs Endothelial Corneal Dystrophy (FECD), which a degenerative disease affecting corneal endothelial cell monolayer Expression of the repeat expansion leads to RNA toxicity and abnormal TCF4 expression through mis-splicing. Most subjects without FECD have between 12 and 40 repeats of a CTG sequence in the third intron of TCF4. In 77.7% of FECD cases in the United States, the CTG repeat sequence measured in peripheral blood leukocytes contains 50 to 3000 repeats.

FECD Is characterized by focal outgrowths and loss of endothelial cells and edema. Because human corneal endothelial cells (hCECs) have minimal regenerative capacity in vivo, reduced hCEC density results in severe cornea damage and loss of vision in advanced FECD cases. Currently the only treatment option is corneal transplantation.

### SUMMARY OF THE INVENTION

According to embodiments of the present invention, there is provided a method of excising an intronic trinucleotide CTG repeat expansion from a Transcription Factor 4 (TCF4) allele in a cell, the method comprising
introducing to the cell a composition comprising:
   at least one CRISPR nuclease, or a nucleotide molecule encoding a CRISPR nuclease; and
   a first RNA molecule comprising a first guide sequence portion, or a nucleotide molecule encoding the first RNA molecule; and
   a second RNA molecule comprising a second guide sequence portion, or a nucleotide molecule encoding the second RNA molecule,
wherein a complex of the CRISPR nuclease and the first RNA molecule affects a double strand break upstream of the intronic trinucleotide CTG repeat expansion and a complex of the CRISPR nuclease and the second RNA molecule affects a double strand break downstream of the intronic trinucleotide CTG repeat expansion,
wherein the first guide sequence portion comprises 17-50 contiguous nucleotides containing nucleotides in the sequence set forth in any one of SEQ ID NOs: 1216-2325 and the second guide sequence portion comprises 17-50 contiguous nucleotides containing nucleotides in the sequence set forth in any one of SEQ ID NOs: 1-1215.

According to embodiments of the present invention, there is also provided a composition comprising an RNA molecule comprising a guide sequence portion having 17-50 contiguous nucleotides containing nucleotides in the sequence set forth in any one of SEQ ID NOs: 1-2325.

According to some embodiments of the present invention, there is also provided a method of treating Fuchs Endothelial Corneal Dystrophy (FECD) in a human subject, the method comprising delivering the any one of the compositions described herein to the subject, or transplanting the any one of the modified cells described herein to a cornea of the human subject.

### BRIEF DESCRIPTION OF THE DRAWINGS

FIG. 1: Schematic representation of Intron 3 of the TCF4 gene. Additionally, a summary table of the depicted TCF4 guides is shown below:

| Name | Guide Sequence Portion |
|---|---|
| hTCF4_g1_20 bp_II | CUCUUCUUCGACGUAUCUAG (SEQ ID NO: 178) |
| hTCF4_g2_20 bp_II | CAGGCAAAUCCUAUACGAGA (SEQ ID NO: 405) |
| hTCF4_g3_20 bp_II | GCAUUUAUUUCGACCCUAAU (SEQ ID NO: 238) |
| hTCF4_g4_20 bp_II | UCCAAAAGAAGGUCUAGAAG (SEQ ID NO: 308) |
| hTCF4_g5_20 bp_II | UGGAGUUUUACGGCUGUACU (SEQ ID NO: 1541) |
| hTCF4_g6_20 bp_II | GCCCCACUUGGAAGGCGGUU (SEQ ID NO: 1436) |
| hTCF4_g7_20 bp_II | UAACUAGGAGGUAAGAUGUA (SEQ ID NO: 1216) |
| hTCF4_g8_20 bp_II | UUGGUAAAUUUCGUAGUCGU (SEQ ID NO: 1575) |

FIG. 2: Activity of sgRNAs targeting Intron 3 of TCF4. RNPs comprised of SpCas9 protein and synthetic RNA guides were electroporated into U2OS cells. Cells were harvested 72 hours post DNA transfection, genomic DNA was extracted, and the TCF4 region targeted by the guide was amplified and analyzed by next-generation sequencing (NGS). The graph represents the % editing±STDV from two independent electroporations introducing the RNPs.

FIG. 3: mRNA levels of TCF4 measured by qRT-PCR. U2OS cells were electroporated with the indicated RNP combinations, RNA was extracted seven (7) days after electroporation, and cDNA was prepared. qRT-PCR was performed with fast SYBR using specific primers for exon 11-exon 12 junction of TCF4 gene. Results are shown as average±STDV (n=3).

FIG. 4: Protein levels of TCF4 measured by western blot (WB). U2OS cells were electroporated with the indicated RNP combinations, protein was extracted (7) days after electroporation following lysis with RIPA. WB was performed with anti-TCF4 and anti-GAPDH antibodies.

FIG. 5: Percent (%) excision measured by ddPCR. U2OS cells were electroporated with the indicated RNP combinations and genomic DNA was extracted seven (7) days after electroporation. ddPCR was performed in QX200 BioRad system, with primers and a probe specific for the excision pattern of g4+g6 (FAM) or RPP30 as a housekeeping gene (HEX). The results show the % of excision calculated from the FAM/HEX ratio (average±STDV, n=3).

FIGS. 6A-6B: Editing and excision of OMNI-103 and OMNI-110 in HeLa cells using DNA transfection. HeLa cells were transfected with plasmids encoding the indicated nuclease and sgRNA. FIG. 6A: Cells were harvested 72 h post DNA transfection, genomic DNA was extracted, and the region of the target guide was amplified and analyzed by NGS. The graph represents the % of editing±STDV from three independent transfection wells. FIG. 6B: Genomic DNA was extracted seven (7) days after electroporation and ddPCR was performed in QX200 BioRad system using EvaGreen, with primers specific for excision events or RPP30 as a housekeeping gene. The results show the % of excision calculated from excision/HKG ratio (avg STDV, n=2).

FIG. 7: Protein levels of TCF4 measured by western blot. HeLa cells were electroporated with the indicated composition, and protein was extracted 14 days after transfection followed by lysis with RIPA. Western blot was performed with anti-TCF4 and anti-GAPDH antibodies.

FIGS. 8A-8B: Editing and excision of OMNI-50 in U2OS cells using LVLPs. FIG. 8A: U2OS cells were infected with downstream and upstream to the repeat compositions. Three (3) days post infection, cells were harvested, genomic DNA was extracted, and the region of the target guide was amplified and analyzed by NGS. FIG. 8B: U2OS cells were infected with either a mix of upstream and downstream composition packed in LVLPs or with LVLPs which contained both compositions in the same particle (all in one). 18 days post infection, cells were harvested for genomic DNA extraction, and excision was measured using ddPCR.

FIG. 9: mRNA levels of TCF4 measured by qRT-PCR after excision with LVLPs. U2OS cells were infected with downstream and upstream to the repeat compositions, RNA was extracted 21 days after electroporation followed by cDNA preparation. qRT-PCR was performed with fast SYBR using specific primers for the exon11-exon12 junction of TCF4 gene. Results shown as average±STDV (n=2).

### DETAILED DESCRIPTION

In healthy individuals, TCF4 is a transcription factor expressed in the corneal endothelium where it regulates the switch between proliferation and differentiation. However, in many Fuchs endothelial corneal dystrophy (FECD) patients, a repeat expansion in TCF4 leads to either toxic RNA formation, dysregulation of TCF4 protein, or toxic protein aggregates (due to repeat-associated non-AUG dependent (RAN) translation), which eventually leads to transcriptional dysfunction and eventually cell death.

Fuchs endothelial corneal dystrophy (FECD) is characterized by formation of guttea-microscopic collagenous aggregates upon the endothelial corneal cells. These aggregates initially lead to blurred vision, which deteriorates over time and can eventually lead to corneal blindness. 70% of FECD subjects in the U.S. carry the TCF4 repeat expansion. The disease is inherited in an autosomal dominant manner.

The present disclosure provides approaches for excising a repeat expansion from at least one TCF4 allele in a cell.

The present disclosure also provides methods of treating, preventing, or ameliorating Fuchs endothelial corneal dystrophy (FECD) by excising a repeat expansion from at least one TCF4 allele in a subject.

In some embodiments, the present disclosure provides a method for utilizing at least one naturally occurring nucleotide difference or polymorphism (e.g., single nucleotide polymorphism (SNP)) for distinguishing/discriminating between two alleles of a gene, one allele bearing a mutation (e.g. a repeat expansion in TCF4) such that it encodes a mutated product causing a disease phenotype (“mutated allele”) and a particular sequence in a SNP position (REF/SNP), and the other allele encoding for a functional product (“functional allele”). In some embodiments, the SNP position is utilized for distinguishing/discriminating between two alleles of a gene bearing one or more disease associated mutations, such as to target one of the alleles bearing both the particular sequence in the SNP position (SNP/REF) and a disease associated mutation. In some embodiments, the disease-associated mutation is targeted. In some embodiments, the method further comprises the step of knocking out expression of the mutated protein and allowing expression of the functional protein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-50, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-2325. Each possibility represents a separate embodiment. In some of these embodiments, the guide sequence portion comprises a sequence that is the same as a sequence set forth in any of SEQ ID NOs: 1-2325. Throughout this application, the terms “guide molecule,” “RNA guide molecule,” “guide RNA molecule,” and “gRNA molecule” are synonymous with a molecule comprising a guide sequence portion. In some embodiments, a molecule comprising a guide sequence portion is a crRNA molecule. In some embodiments, a molecule comprising a guide sequence portion is a crRNA molecule, which is preferably accompanied by a compatible tracrRNA molecule capable of forming a crRNA:tracrRNA complex with the crRNA molecule. In some embodiments, a molecule comprising a guide sequence portion is a sgRNA molecule.

The term “non-discriminatory” as used herein refers to a guide sequence portion of an RNA molecule that targets a specific DNA sequence that is common to all alleles of a gene.

The term “single nucleotide polymorphism (SNP) position”, as used herein, refers to a position in which a single nucleotide DNA sequence variation occurs between members of a species, or between paired chromosomes in an individual. In the case that a SNP position exists at paired chromosomes in an individual, a SNP on one of the chromosomes is a “heterozygous SNP.” The term SNP position refers to the particular nucleic acid position where a specific variation occurs and encompasses both a sequence including the variation from the most frequently occurring base at the particular nucleic acid position (also referred to as “SNP” or alternative “ALT”) and a sequence including the most frequently occurring base at the particular nucleic acid position (also referred to as reference, or “REF”). Accordingly, the sequence of a SNP position may reflect a SNP (i.e. an alternative sequence variant relative to a consensus reference sequence within a population), or the reference sequence itself.

In some embodiments, the present disclosure provides a method for utilizing at least one naturally occurring nucleotide difference or polymorphism (e.g., single nucleotide polymorphism (SNP)) for distinguishing/discriminating between two alleles of a gene, one allele bearing a mutation (e.g. an expanded trinucleotide repeat in TCF4) such that it encodes a mutated product causing a disease phenotype (“mutated allele”) and a particular sequence in a SNP position (SNP/REF), and the other allele encoding for a functional product (“functional allele”). The method further comprises the step of knocking out expression of the mutated protein and allowing expression of the functional protein. In some embodiments, the method is for treating, ameliorating, or preventing a dominant negative genetic disorder.

In accordance with some embodiments, there is provided an RNA molecule which binds to/associates with and/or directs an RNA-guided DNA nuclease to a sequence comprising at least one nucleotide which differs between a mutated allele and a functional allele (e.g., SNP) of a gene of interest (i.e., a sequence of the mutated allele which is not present in the functional allele). The sequence may be within the disease associated mutation. The sequence may be upstream or downstream to the disease associated mutation. Any sequence difference between the mutated allele and the functional allele may be targeted by an RNA molecule of the present invention.

In embodiments of the present invention, an RNA molecule comprises a guide sequence portion having 17-50 contiguous nucleotides containing nucleotides in the sequence set forth in any one of SEQ ID NOs: 1-2325.

The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 17-50 nucleotides in length and contain 20-22 contiguous nucleotides in the sequence set forth in any one of SEQ ID NOs: 1-2325. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-2325. For example, a guide sequence portion having 17 nucleotides in the sequence of 17 contiguous nucleotides set forth in SEQ ID NO: 2326 may consist of any one of the following nucleotide sequences (nucleotides excluded from the contiguous sequence are marked in strike-through):

(SEQ ID NO: 2326)
AAAAAAAUGUACUUGGUUCC
17 nucleotide guide sequence 1:
(SEQ ID NO: 2327) [symbol]
AAAAUGUACUUGGUUCC
17 nucleotide guide sequence 2:
(SEQ ID NO: 2328) [symbol]
AAAAAUGUACUUGGUUC [symbol]
17 nucleotide guide sequence 3:
(SEQ ID NO: 2329) [symbol]
AAAAAAUGUACUUGGUU [symbol]
17 nucleotide guide sequence 4:
(SEQ ID NO: 2330)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-2325 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases. e.g. Cas9, may form a CRISPR complex between the CRISPR nuclease, an RNA molecule, and atracrRNA molecule. A guide sequence portion, which comprises a nucleotide sequence that is capable of hybridizing to a specific target DNA sequence, and a sequence portion that participates in CRISPR nuclease binding, e.g. a tracrRNA sequence portion, can be located on the same RNA molecule. Alternatively, a guide sequence portion may be located on one RNA molecule and a sequence portion that participates in CRISPR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product. as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided a method of excising an intronic trinucleotide CTG repeat expansion from a Transcription Factor 4 (TCF4) allele in a cell, the method comprising
introducing to the cell a composition comprising:
   at least one CRISPR nuclease, or a nucleotide molecule encoding a CRISPR nuclease; and
   a first RNA molecule comprising a first guide sequence portion, or a nucleotide molecule encoding the first RNA molecule; and
   a second RNA molecule comprising a second guide sequence portion, or a nucleotide molecule encoding the second RNA molecule,
wherein a complex of the CRISPR nuclease and the first RNA molecule affects a double strand break upstream of the intronic trinucleotide CTG repeat expansion and a complex of the CRISPR nuclease and the second RNA molecule affects a double strand break downstream of the intronic trinucleotide CTG repeat expansion,
wherein the first guide sequence portion comprises 17-50 contiguous nucleotides containing nucleotides in the sequence set forth in any one of SEQ ID NOs: 1216-2325 and the second guide sequence portion comprises 17-50 contiguous nucleotides containing nucleotides in the sequence set forth in any one of SEQ ID NOs: 1-1215.

In some embodiments, the RNA molecule is a crRNA molecule and the composition further comprises a tracrRNA molecule that forms a crRNA:tracrRNA molecule with the crRNA molecule. In some embodiments, the RNA molecule is an sgRNA molecule.

In some embodiments, the first guide sequence portion comprises 17-50 contiguous nucleotides containing nucleotides in the sequence set forth in any one of SEQ ID NOs: 1216-2325 and the second guide sequence portion comprises 17-50 contiguous nucleotides containing nucleotides in the sequence set forth in any one of SEQ ID NOs: 1-1215 and wherein at least one TCF4 allele in the cell comprises a heterozygous SNP at the rs34071688 position.

In some embodiments, the first guide sequence portion comprises 17-50 contiguous nucleotides containing nucleotides in the sequence set forth in any one of SEQ ID NOs: 1216-2325 and the second guide sequence portion comprises 17-50 contiguous nucleotides containing nucleotides in the sequence set forth in any one of SEQ ID NOs: 1-1215 and wherein at least one TCF4 allele in the cell comprises a heterozygous SNP at any one of the positions selected from the group consisting of rs746872826, 18:5558615, rs879522127. and rs1268568114.

In some embodiments, the first guide sequence portion comprises 17-50 contiguous nucleotides containing nucleotides in the sequence set forth in any one of SEQ ID NOs: 1-1215 and the second guide sequence portion comprises 17-50 contiguous nucleotides containing nucleotides in the sequence set forth in any one of SEQ ID NOs: 1-1215 and wherein at least one TCF4 allele in the cell comprises a heterozygous SNP at any one of the SNP positions selected from the group consisting of rs71670792, rs1261085, rs1261084, rs11431395, rs373174214, rs35555522, rs66807288, rs141970461, rs71674214, rs8766. rs10221362, rs2276195, rs569385112, rs398041379, rs72925008. rs8090085, rs5825130, rs56887277, rs397836157. rs55755941, rs781691419, rs11409023, rs10221357, rs11661961, rs11660565, rs61656112, rs60565673, rs72925018, rs1261073, rs1261076, rs748718974, rs13381800, rs34773632, rs1942265, rs7241077, rs62092440, rs1893431, rs3838898, rs1893430, 18:55251930_G_GTTTT, rs771385941, rs4800988, rs1942264, rs1261093, rs1539950, rs1539951, rs62092442, rs62092444, rs11662842, rs11664992, rs1261134, rs1261114, rs1788027, rs397858367, rs113662542, rs1261118, rs10701336, rs397809469, rs150323043, rs1038226655, rs149454001, rs796169498, rs9955026, rs1046741326, rs754435392, rs867471715, rs777608256, rs762153709, rs1153636, rs1153637, rs5825134. rs35480166, rs893946, rs374155330, rs781053385. rs899293868, rs996440450, rs1349129287. rs780424692, rs34702622. rs11428164, rs1660235, rs1440473, rs1788026, rs11332509, rs1660237, rs1631486, rs1025804, rs753933037, rs1660233, rs200650987, rs71951255, rs368762262, rs751007744, rs780342991, rs1660241, rs796749696, rs61468075, rs777518462, rs1660242, rs1440476. rs1011392, 18:55374871_T_TA, rs1788030, rs1623427, rs1621581, rs1788025. rs1788023, rs1348047. rs1788019, rs9950000, rs9958125, rs9320010, rs3794891, rs757629087, rs772409228, rs12607679, rs3794889, rs4801149, rs12605773, rs2872041, rs4801150, rs1020169, rs7238888, rs7235757, rs2958178, rs2958165, rs2958171, rs1328839434, rs796792902, rs2958175, rs11309751, rs2958186, rs2919446, rs2958161, rs2860511, rs2958162, rs2919451, rs2919450, rs201657057, rs2958163. rs1440477, rs2958166, rs377458803, rs2958169, rs8098843. rs11412305, rs386387765, 18:55423173_T_TA, rs781071274, rs4374254, rs370693034, rs761981780, rs8084308, rs77540208, rs9320016, rs4524013, rs1025639279, rs4500831, rs7229456, rs12967143, rs12963334, rs12963463, rs398100891, rs12958048, 18:55434419_C_CTTT, rs375388593, rs140134419, rs4801153, rs4801154, rs745460290, rs527450659, rs4341827, rs4468713, rs7228159, rs145330990, rs7231748, rs34577882, rs34578042, rs1452789, rs1452788, rs12606995, rs188225813, rs732779, rs11385247, rs9966430, rs2924321, rs151196106, rs3760600, rs2924328, rs1377243, rs2924329, rs5825142, rs199707137, 18:55468891_C_CCCA, rs11338618, rs149728054, rs11298284, rs7233312, rs2924331, 18:55480276_C_CAA, rs2958182, rs2958183, rs2958184, rs2924332, rs2924333, rs2060889, rs2958187, rs2924335, rs138885827, rs2924336, rs59413482, rs796565215, 18:55496896_C_CAA, rs4801157, rs2958188, rs2958189, rs2060886, rs3017183, rs2958158, rs2924338, rs12956276, rs55812411, rs776881842. rs1452791. rs9957668, rs9954890, rs9964328, rs67387556, rs1491335073, 18:55511330_T_TAAA, rs751932079, rs2957261, 18:55511331_T_TAAAA, rs8090106, rs140221855, rs17089851, rs398032944, rs1341922999, rs624244, rs627685, rs9948513, rs9965067, rs9965195, rs35371867, rs11441646, rs9949107, rs7240986, rs4801158, rs72627231, rs11412432, rs33938531, rs4800990, rs4458089, rs4572488, rs12968271, rs9636107. rs2123389, rs9947814, rs71352207, rs1452787, rs2123392. rs2123393, 18:55559041_T_TA, rs34935191, rs74182105, rs139870092, rs76053687, rs150848781. rs564960433, rs41396445, and rs34232463.

In some embodiments, the composition is introduced to a cell in a subject or to a cell in culture.

In some embodiments, the cell is a corneal cell or a corneal endothelial cell.

In some embodiments, the composition is introduced to the cell in vivo.

In some embodiments, the composition is introduced to the cell by a lentivirus-like particle (LVLP).

In some embodiments, the cell is a stem cell, a fibroblast, blood cell, hepatocyte, keratinocyte, any other cell type capable of being reprogrammed to an induced pluripotent stem cell (iPSC), a hematopoietic stem cell (HSC), an induced pluripotent stem cell (iPS cell), an iPSc-derived cell, or a iPSc-derived corneal endothelial cell.

In some embodiments, the composition is introduced to the cell ex vivo.

In some embodiments, the CRISPR nuclease, the first RNA molecule, and the second RNA molecule are introduced to the cell at substantially the same time or at different times.

In some embodiments, the first or second RNA molecule is a crRNA molecule or a sgRNA molecule.

According to embodiments of the present invention, there is provided a composition comprising an RNA molecule having a guide sequence portion that comprises 17-50 contiguous nucleotides containing nucleotides in the sequence set forth in any one of SEQ ID NOs: 1-2325.

In some embodiments, the composition further comprises a second RNA molecule, wherein the first RNA molecule has a guide sequence portion that comprises 17-50 contiguous nucleotides containing nucleotides in the sequence set forth in any one of SEQ ID NOs: 1216-2325, and the second RNA molecule has a guide sequence portion that comprises 17-50 contiguous nucleotides containing nucleotides in the sequence set forth in any one of SEQ ID NOs: 1-1215.

In some embodiments, the composition further comprises a CRISPR nuclease.

In some embodiments, the composition further comprises a tracrRNA molecule.

According to embodiments of the present invention, there is provided a cell modified by any one of the disclosed methods, or a cell modified using any one of the disclosed compositions.

In some embodiments, the cell is a corneal cell or corneal endothelial cell.

In some embodiments, the cell is a stem cell, or a fibroblast, blood cell, hepatocyte, keratinocyte, or any other cell type capable of being reprogrammed to an induced pluripotent stem cell (iPSC), a hematopoietic stem cell (HSC), iPSC-derived cell, or a iPSC-derived corneal endothelial cell.

In some embodiments, the stem cell is differentiated after it is modified.

In some embodiments, the stem cell is differentiated into a corneal cell or a corneal endothelial cell.

According to embodiments of the present invention, there is provided a method of treating Fuchs Endothelial Corneal Dystrophy (FECD) in a human subject. the method comprising delivering any one of the compositions described herein to the subject, or transplanting any one of the modified cells described herein to a cornea of the human subject.

In some embodiments, the composition is delivered to a cell in the cornea of a patient in vivo.

In some embodiments, the composition is introduced to the cell by a lentivirus-like particle (LVLP).

In some embodiments, the modified cell is delivered to a cell in the cornea of a patient ex vivo.

According to embodiments of the present invention, there is provided a medicament comprising the any one of the compositions described herein for use in excising an intronic trinucleotide CTG repeat expansion from a TCF4 allele in a cell, wherein the medicament is administered by delivering to the cell the composition.

According to embodiments of the present invention, there is provided use of any one of the compositions described herein or any one of the modified cells described herein for treating FECD. comprising delivering the composition or the modified cell to a subject having or at risk of having FECD.

According to embodiments of the present invention, there is provided a medicament comprising any one of the compositions described herein or any one of the modified cells described herein for use in treating FECD, wherein the medicament is administered by delivering the composition or the modified cell to a subject having or at risk of having FECD.

According to embodiments of the present invention, there is provided a kit for excising an intronic trinucleotide CTG repeat expansion from a TCF4 allele in a cell, comprising any one of the compositions described herein and instructions for delivering the composition to the cell.

In some embodiments, the composition is delivered to the cell ex vivo.

According to embodiments of the present invention, there is provided a kit for administering treating FECD in a subject, comprising any one of the compositions described herein or any one of the modified cells described herein and instructions for delivering the composition or modified cell to a subject having or at risk of having FECD.

According to embodiments of the present invention, there is provided any one of the compositions described herein or any one of the modified cells described herein for use in treating FECD. comprising delivering the composition or the modified cell to a subject having or at risk of having FECD.

In some embodiments, the first or second RNA molecules recited in the methods and compositions described herein may be modified to contain 1, 2, 3, 4, or 5 nucleotide mismatches in its guide sequence portion relative to its intended target sequence. In some embodiments, these mismatches provide higher targeting specificity to a complex of the CRISPR nuclease and the RNA molecule compared to the specificity provided by a guide sequence portion that has higher complementarity to its intended target sequence.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-2325. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for excising an intronic trinucleotide CTG repeat expansion from a Transcription Factor 4 (TCF4) allele in a cell, the method comprising delivering to the cell a composition comprising an RNA molecule comprising a guide sequence portion having 17-50 contiguous nucleotides containing nucleotides in the sequence set forth in any one of SEQ ID NOs: 1216-2325 and a CRISPR nuclease and a second RNA molecule comprising a guide sequence portion having 17-50 contiguous nucleotides containing nucleotides in the sequence set forth in any one of SEQ ID NOs: 1-1215 and a CRISPR nuclease.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

The compositions and methods of the present disclosure may be utilized for Fuchs Endothelial Corneal Dystrophy (FECD) therapy.

Any one of, or combination of, the above-mentioned strategies for deactivating TCF4 expression may be used in the context of the invention.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a cell in the cornea. In some embodiments, the cell is a corneal endothelial cell. In some embodiments, the cell is a stem cell, or a fibroblast, blood cell, hepatocyte, keratinocyte, or any other cell type capable of being reprogrammed to an induced pluripotent stem cell (iPSC).

### TCF4 Editing Strategies to Excise a CTG Expanded Repeat

The TCF4 gene encodes Transcription Factor 4, a basic helix-loop-helix transcription factor. The encoded protein recognizes an Ephrussi-box (‘E-box’) binding site (‘CANNTG’)—a motif first identified in immunoglobulin enhancers. This gene is broadly expressed and may play an important role in nervous system development. Defects in this gene are a cause of Pitt-Hopkins syndrome. Multiple alternatively spliced transcript variants that encode different proteins have been described.

Furthermore, a TCF4 intronic CTG repeat normally numbering 10-37 repeat units can expand to >50 repeat units and cause disease. Specifically, Fuchs Endothelial Corneal Dystrophy causative mutations are heterozygous TCF4 intronic trinucleotide repeat expansions (CTG)n. The CTG repeats are bidirectionally transcribed leading to the disease, therefore, a therapeutic strategy involves excision of the CTG repeats from a TCF4 allele.

Thus, the present invention provides methods to excise a CTG three nucleotide repeat (TNR) from at least one TCF4 allele using two guide molecules. The repeat is located in TCF4 intron 3, which begins at 18:55587044 and ends at 18:55585353.

One strategy is to perform biallelic excision of the TCF4 repeats. this can be achieved by utilizing guides adjacent to the expanded repeat in TCF4 intron 3. For example, a first guide may target a region of hg38_chr18:55585922-55586155 (downstream to the repeat) and a second guide may target a region of hg38_chr18: 55586227-55586483 (upstream to the repeat) to excise the expanded TCF4 repeat.

Monoallelic excision of a TCF4 expanded repeat can be achieved by utilizing a first guide targeting a SNP located in intron 3, upstream or downstream to the expanded repeat, and a second, non-discriminatory guide targeting a sequence in intron 3. For example, excision of an TCF4 expanded repeat may be performed by using a first guide to target the SNP position rs34071688, which is located upstream to the repeat, and a second, non-discriminatory guide targeting a sequence located downstream to the repeat.

Monoallelic excision of a TCF4 expanded repeat be can also be achieved by excising the expanded repeat while knocking-out the allele with the expanded repeat. This can be done by utilizing guides targeting SNPs located in different regions of the gene such as, exons, introns, promoter regions or in intergenic regions, downstream or upstream to the gene. The non-discriminatory guide should be located in intron 3 to enable the excision of the gene fragment including the CTG repeats. This strategy would lead to knock-out the allele with the expanded repeat due to excising large fragments that would prevent its transcription, destabilize the transcript, generate an immature stop codon, which would lead to nonsense meditated decay or to a truncated protein.

In some embodiments, one of the guide molecules targets upstream to the TNR, and the other guide molecule targets downstream to the TNR. Excision of the TNR without altering TCF4 normal expression can be achieved, for example, by excision of CTG TNR by two guides targeting up to 350 nucleotides upstream and up to 250 nucleotides downstream to the TNR.

In some embodiments, the target cell is a cell in the cornea. In some embodiments, the cell is a corneal endothelial cell. In some embodiments, delivery of the guide molecules is performed in vivo. In some embodiments, the delivery is performed using a lentivirus-like particle (LVLP).

Alternatively, cells such as iPS derived endothelial cells can be edited ex vivo and transplanted to the cornea, or iPS cells can be edited and then differentiated and transplanted to the cornea.

### CRISPR Nucleases and PAM Recognition

In some embodiments, the sequence specific nuclease is selected from CRISPR nucleases, or is a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to all TCF4 alleles in a cell. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant: NGCG for SpCas9-VRER variant: NGAG for SpCas9-EQR variant: NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase: NG or NA for SpCas9-NG variant of SpCas9 wherein N is any nucleobase: NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase: TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides. Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium dificile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRISPR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRISPR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

In some embodiments, the CRISPR nuclease or derivative thereof may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity. deacetylase activity. kinase activity, phosphatase activity, ubiquitin ligase activity, deubiquitinating activity. adenylation activity. deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity deacetylase activity, kinase activity, phosphatase activity, ubiquitin ligase activity or deubiquitinating activity.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine. uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-O-methyl (M), 3′-phosphorothioate (MS). 3′-thioPACE (MSP), pseudouridine, or 1-methyl pseudo-uridine. Each possibility represents a separate embodiment of the present invention.

In addition to targeting TCF4 alleles by a RNA-guided CRISPR nuclease, other means of inhibiting TCF4 expression in a target cell include but are not limited to use of a gapmer, shRNA, siRNA, a customized TALEN, meganuclease, or zinc finger nuclease. a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736: WO/2006/097853; WO/2003/087341; WO/2000/0415661: WO/2003/080809; WO/2010/079430; WO/2010/079430: WO/2011/072246; WO/2018/057989; and WO/2017/164230, the entire contents of each of which are incorporated herein by reference.

Advantageously, the guide RNA molecules presented herein provide improved TCF4 knockout efficiency when complexed with a CRISPR nuclease in a cell relative to other guide RNA molecules. These specifically designed sequences may also be useful for identifying TCF4 target sites for other nucleotide targeting-based gene-editing or gene-silencing methods, for example, siRNA, TALENs, meganucleases or zinc-finger nucleases.

### Delivery to Cells

Any one of the compositions described herein may be delivered to a target cell by any suitable means. RNA molecule compositions of the present invention may be targeted to any cell which contains and/or expresses a TCF4 allele, such as a corneal endothelial cell or stem cell. The delivery to the cell may be performed in vivo, ex vivo, or in vitro. In some embodiments, a lentivirus like particle (LVLP) is used to deliver the composition.

Further, the nucleic acid compositions described herein may be delivered to a cell as one or more of DNA molecules, RNA molecules, ribonucleoproteins (RNP), nucleic acid vectors, or any combination thereof.

In some embodiments, the RNA molecule comprises a chemical modification. Non-limiting examples of suitable chemical modifications include 2′-O-methyl (M), 2-O-methyl, 3′phosphorothioate (MS) or 2′-O-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993): Miller (1992); Van Brunt (1988): Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus. adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70. and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into cells include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

Examples of RNA Guide Sequences which Specifically Target Alleles of TCF4 Gene

Although a large number of guide sequences can be designed to target the TCF4 gene, the nucleotide sequences described in Table 1 and are identified by SEQ ID NOs: 1-2325 were specifically selected to effectively implement the methods set forth herein.

Table 1 shows guide sequences designed for use as described in the embodiments above to associate with target sequences of the TCF4 gene. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN). SpCas9.VQR.2 (PAM SEQ: NGNG). SpCas9 EQR (PAM SEQ: NGAG). SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), JeCas9WT (PAM SEQ NNN VRYM), OMNIT-50 (PAM SEQ: NGG), OMNI-79 (PAM SEQ NGG), OMNI-103 (PAM SEQ: NNRACT), OMNI-159 (NNNNCMAN), or OMNI-124 (PAM SEQ: NNGNRMNN). RNA molecules of the present invention are each designed to form complexes in conjunction with one or more different CRISPR nucleases and designed to target polynucleotide sequences of interest utilizing one or more different PAM sequences respective to the CRISPR nuclease utilized.

Additional description of OMNI CRISPR nucleases is provided in PCT International Application Publication No. WO 2023/019269 A2, PCT International Application Publication Nos. WO 2022/170199 A2 and WO 2023/107946 A2, U.S. Pat. No. 11,666,641 B2 and PCT International Application Publication Nos. WO 2020/223514 A2, WO 2022/098693 A1, and WO 2023/019263 A1, U.S. Application Publication No. 2023/0122086 A1 and PCT International Application Publication Nos. WO 2021/248016 A2 and WO 2023/102407 A2, PCT International Application Publication No. WO 2022/087135 A1, and PCT International Application Publication No. WO 2022/226215 A1, the contents of each of which are hereby incorporated by reference.

As used herein, the following nucleotide identifiers are used to represent a referenced nucleotide base(s):

| Nucleotide | | | | |
|---|---|---|---|---|
| reference | Base(s) represented | | | |
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

TABLE 1
Guide sequences designed to associate with specific TCF4 gene targets

| | SEQ ID NOS: | SEQ ID NOS: | SEQ ID NOS: |
|---|---|---|---|
| | of 20 base | of 21 base | of 22 base |
| Target | guides | guides | guides |
| 18:55585911-55586155 | 1-405 | 406-809 | 810-1215 |
| Intron 3, downstream to the expanded | | | |
| region | | | |
| 18:55586227-55586483 | 1216-1604 | 1605-1958 | 1959-2325 |
| Intron 3, upstream to the expanded | | | |
| region | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12. The SNP details are indicated by the listed SNP ID Nos. (“rs numbers”), which are based on the NCBI 2018 database of Single Nucleotide Polymorphisms (dbSNP)). The indicated DNA mutations are associated with Transcript Consequence NM_001083962 as obtained from NCBI RefSeq genes

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: TCF4 Guide Sequence Portion Targeting Analysis

Guide sequences comprising 17-50 contiguous nucleotides containing nucleotides in the sequence set forth in any one of SEQ ID NOs: 1-2325 are screened for high on target activity using a CRISPR nuclease human cells. On target activity is determined by DNA capillary electrophoresis analysis.

### Example 2: TCF4 Expanded Repeat Excision

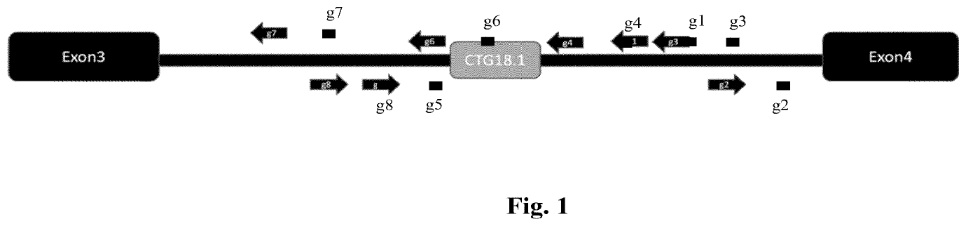

Trinucleotide repeat CTG18.1 is located in intron 3 of the TCF4 gene. In up to 75% of FECD patients, at least one TCF4 allele with CTG18.1 expansion (i.e. over 50 copies of “CTG”) is present. In order to perform excision of the repeat for use in FECD treatment, four (4) different sgRNAs were designed up and downstream to the repeat (see FIG. 1), and the activity of each sgRNA was tested with SpCas9 in U2OS cells.

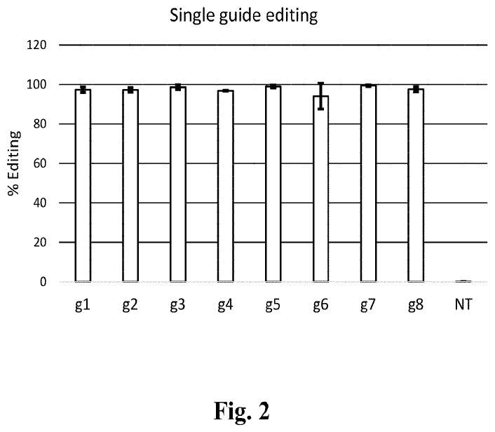

Briefly, 2×10⁵ cells were mixed with preassembled RNPs, which were composed of 105 pmole SpCas9 protein (Alt-R® S.p. Cas9 Nuclease V3, 1081059, IDT) and 124 pmole sgRNA (Alt-R™ CRISPR-Cas9 sgRNA. IDT), and 100 pmole of electroporation enhancer (Alt-R® Cas9 Electroporation Enhancer, 1075916). The cells were then electroporated using SE cell 4D-nucleofector X Kit S (V4XC-1032, Lonza) by applying the DN-100 program. A fraction of the cells was harvested 72 hours post-electroporation and genomic DNA was extracted to measure on-target activity by next generation sequencing (NGS). According to NGS analysis, all eight (8) guides demonstrated very high activity and editing was observed in over 90% of the cells (FIG. 2).

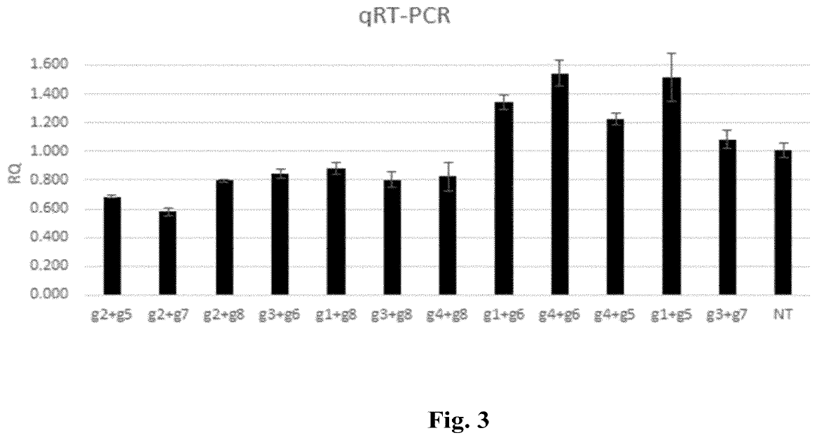

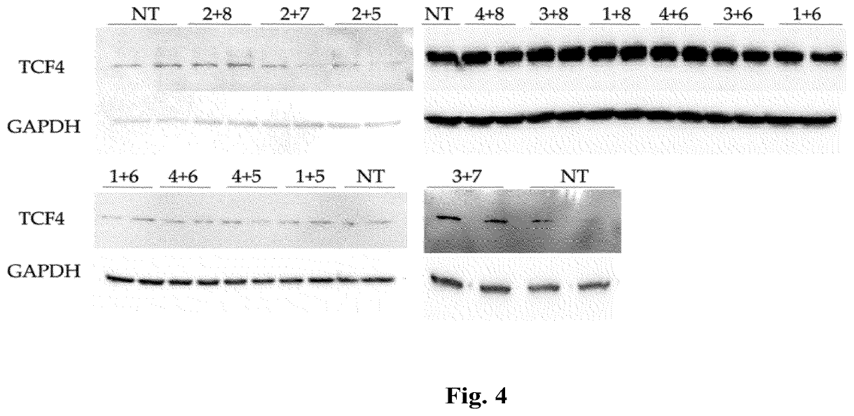

In order to see that elimination of the repeat area does not affect TCF4 expression, excision of the repeat area with different guide combinations (upstream and downstream to the repeat) was performed in U2OS cells and the expression of TCF4 was tested using qRT-PCR and western blot. Briefly, 2×10⁵ cells were mixed with preassembled RNPs composed of 105 pmole SpCas9 protein (Alt-R® S.p. Cas9 Nuclease V3, 1081059, IDT) and 124 pmole of each sgRNA (Alt-R™ CRISPR-Cas9 sgRNA. IDT). Upon RNP formation, the indicated RNP combinations were mixed with 100 pmole of electroporation enhancer (Alt-RC Cas9 Electroporation Enhancer, 1075916) and electroporated using SE cell 4D-nucleofector X Kit S (V4XC-1032, Lonza) by applying the DN-100 program. Cells were allowed to grow for at least seven (7) days in order to obtain enough cells for genomic DNA. RNA and protein extraction. Upon RNA extraction, cDNA was synthesized, and qRT-PCR was performed using fast SYBR and specific primers to the exon 11-exon 12 junction of TCF4 (FIG. 3). For western blot, cells were lysed with RIPA buffer and 40 μg of total protein was separated using SDS-PAGE. A specific TCF4 antibody (ab217668, Abcam) was used to detect protein levels of TCF4. GAPDH was used as a loading control (FIG. 4).

As shown, only excision with gRNA2+gRNA5 and gRNA2+gRNA7 reduced the levels of mRNA and protein of TCF4.

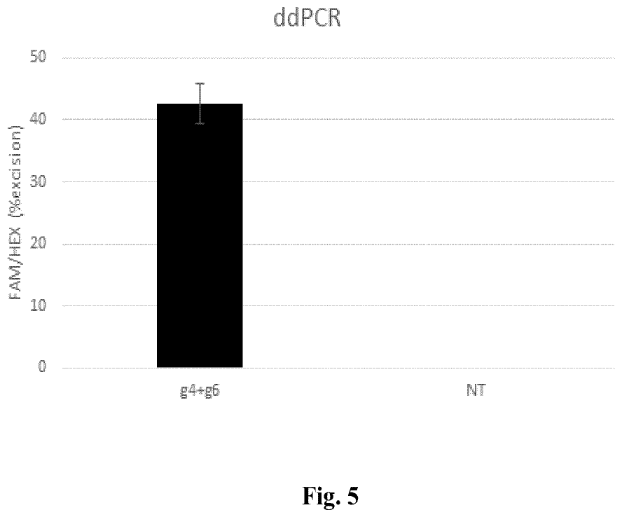

In order to validate the excision levels, a ddPCR assay (QX200. BioRad) was designed with a probe specific to the excision pattern of g4+g6 so that only the excised fragments can give a positive signal (FAM). RPP30 was used as an endogenous control (HEX). The results show that the excision rate was 42% for the g4+g6 combination (FIG. 5).

A summary table of the tested TCF4 guide sequence portions is shown below:

| Name | Guide Sequence Portion |
|---|---|
| hTCF4_g1_20 bp_II | CUCUUCUUCGACGUAUCUAG (SEQ ID NO: 178) |
| hTCF4_g2_20 bp_II | CAGGCAAAUCCUAUACGAGA (SEQ ID NO: 405) |
| hTCF4_g3_20 bp_II | GCAUUUAUUUCGACCCUAAU (SEQ ID NO: 238) |
| hTCF4_g4_20 bp_II | UCCAAAAGAAGGUCUAGAAG (SEQ ID NO: 308) |
| hTCF4_g5_20 bp_II | UGGAGUUUUACGGCUGUACU (SEQ ID NO: 1541) |
| hTCF4_g6_20 bp_II | GCCCCACUUGGAAGGCGGUU (SEQ ID NO: 1436) |
| hTCF4_g7_20 bp_II | UAACUAGGAGGUAAGAUGUA (SEQ ID NO: 1216) |
| hTCF4_g8_20 bp_II | UUGGUAAAUUUCGUAGUCGU (SEQ ID NO: 1575) |

### Example 3: Guide Sequence Portion Screening

Additional guides were screened using several OMNI CRISPR nucleases in HeLa cells. This is a proof of concept showing that guides can form double-stranded breaks upstream and downstream to the TCF4 repeat expansion.

A summary table of the additionally tested TCF4 guide sequence portions is shown below. Briefly, sgRNA activity targeting TCF4 intron 3 around the expanded repeat CTG18.1 locus is shown. In order to test activity of each guide with the indicated nuclease, HeLa cells were transfected with both a nuclease and the sgRNA and activity was measured three (3) days after transfection using next-generation sequencing (NGS).

| | | | | | Upstream/ |
|---|---|---|---|---|---|
| | | | | | Downstream |
| | | | | | to the |
| CRISPR | Guide | Guide Sequence | | % Max | Repeat |
| Nuclease | Name | Portion | PAM | Edits | Expansion |
| OMNI-110 | g128 | AGCAAAGGGATGG | AAGTACAG | 54.1 | Upstream |
| | | AGAAGGACC (SEQ | | | |
| | | ID NO: 2026) | | | |
| | g189 | GTCCCAGACATGTC | GAATTCAT | 71.52 | Upstream |
| | | AGGAGAAT (SEQ ID | | | |
| | | NO: 2200) | | | |
| | g51 | TCTGACTCAGGGA | TTATCCAC | 42.4 | Downstream |
| | | AGGTGTGCA (SEQ | | | |
| | | ID NO: 1141) | | | |
| | g183 | GGGAAGGTGTGCA | AGATACGT | 68.7666667 | Downstream |
| | | TTATCCACT (SEQ | | | |
| | | ID NO: 1078) | | | |
| | g185 | CTTTCTGCTTGTTG | CCATTCGT | 60.7533333 | Downstream |
| | | CACTTTCT (SEQ ID | | | |
| | | NO: 1007) | | | |
| OMNI-231 | g180 | GGATCAGCACAAA | GACACACT | 60.02 | Upstream |
| | | GCGGAACTT (SEQ | | | |
| | | ID NO: 2180) | | | |
| | g181 | TTCGTAGTCGTAGG | AAAGCGGA | 58.5633333 | Upstream |
| | | ATCAGCAC (SEQ ID | | | |
| | | NO: 2285) | | | |
| | g67 | GTTTGGTGTAAGAT | AAAGCAAA | 58.3766667 | Upstream |
| | | GCATTTGT (SEQ ID | | | |
| | | NO: 2212) | | | |
| | g14 | GAGAAGGACCAAG | AAAACTCC | 42.2666667 | Upstream |
| | | TACAGCCGT (SEQ | | | |
| | | ID NO: 2151) | | | |
| OMNI-269 | g61 | GCTGATCCTACGAC | TTACCAAA | 80.0033333 | Upstream |
| | | TACGAAAT (SEQ ID | | | |
| | | NO: 2167) | | | |
| | g67 | GTTTGGTGTAAGAT | AAAGCAAA | 58.025 | Upstream |
| | | GCATTTGT (SEQ ID | | | |
| | | NO: 2212) | | | |
| | g170 | AATCCACAAAACA | AATCCAAA | 49.5266667 | Upstream |
| | | CACAAATAA (SEQ | | | |
| | | ID NO: 2325) | | | |
| | g59 | ACAGCCGTAAAAC | GTGTCAAG | 54.49 | Upstream |
| | | TCCACAAGT (SEQ | | | |
| | | ID NO: 2007) | | | |
| | g66 | GAATCCACAAAAC | AAATCCAA | 60.91 | Upstream |
| | | ACACAAATA (SEQ | | | |
| | | ID NO: 2144) | | | |
| | g49 | CTAGATACGTCGA | AAACCAAT | 70.3766667 | Downstream |
| | | AGAAGAGGG (SEQ | | | |
| | | ID NO: 973) | | | |
| | g41 | TTCCCTGAGTCAGA | AAAGCAAA | 69.1666667 | Downstream |
| | | GCCTGCAA (SEQ ID | | | |
| | | NO: 1173) | | | |
| OMNI-129 | g141 | CCGCTTTGTGCTGA | CTACGAAA | 54.31 | Upstream |
| | | TCCTACGA (SEQ ID | | | |
| | | NO: 2103) | | | |
| OMNI-136 | g41 | TTCCCTGAGTCAGA | AAAGCAAA | 55.0233333 | Upstream |
| | | GCCTGCAA (SEQ ID | | | |
| | | NO: 1173) | | | |
| OMNI-169 | g131 | AGGGATGGAGAAG | CAGCCGTA | 53.5233333 | Upstream |
| | | GACCAAGTA (SEQ | | | |
| | | ID NO: 2036) | | | |
| OMNI-286 | g67 | GTTTGGTGTAAGAT | AAAGCAAA | 67.87 | Upstream |
| | | GCATTTGT (SEQ ID | | | |
| | | NO: 2212) | | | |
| OMNI-159 | g40 | GCACACCTTCCCTG | CCTGCAAA | 48.95 | Downstream |
| | | AGTCAGAG (SEQ ID | | | |
| | | NO: 1040) | | | |
| | g42 | AAGCAAAGGAACG | AGTGCAAC | 42.99 | Downstream |
| | | AATGGAGAA (SEQ | | | |
| | | ID NO: 830) | | | |
| | g43 | CAAAGGAACGAAT | GCAACAAG | 52.49 | Downstream |
| | | GGAGAAAGT (SEQ | | | |
| | | ID NO: 907) | | | |
| | g49 | CTAGATACGTCGA | AAACCAAT | 41.35 | Downstream |
| | | AGAAGAGGG (SEQ | | | |
| | | ID NO: 973) | | | |
| | g59 | ACAGCCGTAAAAC | GTGTCAAG | 43.9 | Upstream |
| | | TCCACAAGT (SEQ | | | |
| | | ID NO: 2007) | | | |
| | g67 | GTTTGGTGTAAGAT | AAAGCAAA | 41.29 | Upstream |
| | | GCATTTGT (SEQ ID | | | |
| | | NO: 2212) | | | |
| OMNI-103 | g10 | GCCTAGGGCTACG | AAAACTTC | 49.21 | Downstream |
| | | TTTCCTGGC (SEQ | | | |
| | | ID NO: 1053) | | | |
| | g13 | GCTTTACAAATGCA | CAAACTCA | 68.1366667 | Upstream |
| | | TCTTACAC (SEQ ID | | | |
| | | NO: 2170) | | | |
| | g14 | GAGAAGGACCAAG | AAAACTCC | 59.29 | Upstream |
| | | TACAGCCGT (SEQ | | | |
| | | ID NO: 2151) | | | |
| | g15 | AGTTCCGCTTTGTG | ACGACTAC | 52.4366667 | Upstream |
| | | CTGATCCT (SEQ ID | | | |
| | | NO: 2041) | | | |
| | g17 | CAATCCAAAGCAT | AAAACTTT | 62.5133333 | Upstream |
| | | CATTAGCTT (SEQ | | | |
| | | ID NO: 2319) | | | |
| | g18 | ATTAGCTTAAAACT | ACAACTGG | 62.0233333 | Upstream |
| | | TTAAAGAG (SEQ ID | | | |
| | | NO: 2320) | | | |
| | g19 | TAGGGAATTGAAG | AAAACTCA | 58.61 | Upstream |
| | | GCCAGTAAT (SEQ | | | |
| | | ID NO: 2321) | | | |
| | g20 | AGTCGTAGGATCA | GGAACTTG | 56.5366667 | Upstream |
| | | GCACAAAGC (SEQ | | | |
| | | ID NO: 2039) | | | |
| | g21 | TACATCTTTTCCTA | AAAACTAG | 54.4133333 | Upstream |
| | | GGATTCTT (SEQ ID | | | |
| | | NO: 2322) | | | |
| | g23 | CCCCTAAAACTAA | AAAACTTG | 86.21 | Upstream |
| | | ACCACCCCT (SEQ | | | |
| | | ID NO: 2323) | | | |
| | g24 | GAAAGCAAAAATA | AAAACTAA | 58.1333333 | Upstream |
| | | GACACCCCT (SEQ | | | |
| | | ID NO: 2324) | | | |
| OMNI-127 | g26 | TTATCAAGATTCAG | CCAGCAGC | 44.7166667 | Downstream |
| | | GTTGGAGG (SEQ ID | | | |
| | | NO: 1214) | | | |
| | g27 | TCAAGATTCAGGTT | GCAGCCTC | 59.2466667 | Downstream |
| | | GGAGGCCA (SEQ ID | | | |
| | | NO: 1215) | | | |
| | g33 | AAGGGATGGAGAA | ACAGCCGT | 54.515 | Upstream |
| | | GGACCAAGT (SEQ | | | |
| | | ID NO: 1986) | | | |
| OMNI-50 | g4 | TCTCCAAAAGAAG | AGGAGGAG | 51.09 | downstream |
| | | GTCTAGAAG (SEQ | | | |
| | | ID NO: 1138) | | | |
| | g195 | AAATCCAAACCGC | GGGGCTCT | 52.92 | upstream |
| | | CTTCCAAGT (SEQ | | | |
| | | ID NO: 1970) | | | |

A table describing the amino acid sequences of the CRISPR nucleases used and the sequences of their sgRNA scaffolds is shown below:

| CRISPR Nuclease | sgRNA Scaffold Sequence |
|---|---|
| OMNI-110 | GUUGUGAUUCGCUUCCGAAAGCAAGCGAAUCACAAUAAGGAUUA |
| (SEQ ID NO: 2331) | UUCCGUUGUGAAAACAUUUAAGUCGGGCCUCCUUCGGUUGGCUC |
| | GGCUUUUUUU (SEQ ID NO: 2342) |
| OMNI-231 | GUUUGAGAGUAAUGUAGGAAAUUACAUUACAAGUUCAAAUAACG |
| (SEQ ID NO: 2332) | AUUUAAUCGAAACCACCUUUUUAGGUACUGCGGUUGCAGUUUUU |
| | U (SEQ ID NO: 2343) |
| OMNI-269 | GCUAUAGUUUCCUUUCGAAGAAAUUCGAAACGUUACUAUAGUAA |
| (SEQ ID NO: 2333) | GAAAUUUUCGAAAAGUUCUGCCUAAUACUAUUAUGUAUUAGGCA |
| | UCUUUUUU (SEQ ID NO: 2344) |
| OMNI-129 | GUUGUAGUUCCCUAAUGUUGAAAGACAUUAGGUUACUGCGAUCA |
| (SEQ ID NO: 2334) | GGCAGUAUGCCUCAGAGCUCCGCCCUAACCACGUUUUGUGGUUG |
| | GGGCGUCUUUGCAUUUUUU (SEQ ID NO: 2345) |
| OMNI-136 | GUUGUAGUUCCCUGUUAAUGAAAAUUUUCGGGUUACUAUGAUAA |
| (SEQ ID NO: 2335) | GGUAGAACACCGAAAAGCUCUAACGCCUUGCCAUUUGGUGAGGC |
| | GUUAUCUUUUUU (SEQ ID NO: 2346) |
| OMNI-169 | GUUGUGAAUUGCUUUCAAAGAAAUGUGAAAGCUUUUCACAAUAA |
| (SEQ ID NO: 2336) | GGCUAUAAGCCACAGAUCUUUCUAACUCCUGCGUACUCCGUGGG |
| | AGUAUUUUUU (SEQ ID NO: 2347) |
| OMNI-286 | GUUGUAGUUCCCUAUUUAUGAAAGUAAAUAGGUUACUACAAUAA |
| (SEQ ID NO: 2337) | GGCCCUUGCGCAAGCAUGGUGCCGCAACUGGAAGGUGCUGUGCG |
| | AGUCACGGCACUUUUUU (SEQ ID NO: 2348) |
| OMNI-159 | GUUGUAGUUCCCUAUUUGUGAAAACAAUUAGGUUACUAUGAUAA |
| (SEQ ID NO: 2338) | GGUAGUAUACCGCAAAGCUCUAACGCCCCGUCUUUGACGGGGCG |
| | UUAUCUUUUUU (SEQ ID NO: 2349) |
| OMNI-103 | GUUUGAGAGUAGUGUAAGAAAUUACACUACAAGUUCAAAUAAAA |
| (SEQ ID NO: 2339) | AUUUAUUCAAAUCCAUUUGCUACAUUGUGUAGAAUUUAAAGAUC |
| | UGGCAACAGAUCUUUUUUU (SEQ ID NO: 2350) |
| OMNI-127 | GUUUUGUUACCAUAUGGAUGAAAAUCUAUAUGACCUAACAAAAC |
| (SEQ ID NO: 2340) | AAGGGUUUAUCCCGGAUUCGGCUCCUUUAUAGGAGCCUUUUUU |
| | (SEQ ID NO: 2351) |
| OMNI-50 | GUUUGAGAGUUAUGGAAACAUGACGAGUUCAAAUAAAAAU |
| (SEQ ID NO: 2341) | UUAUUCAAACCGCCUAUUUAUAGGCCGCAGAUGUUCUGCAU |
| | UAUGCUUGCUAUUGCAAGCUUUUUU (SEQ ID NO: 2352) |

### Example 4: Excision Evaluation—Transfection in HeLa Cells

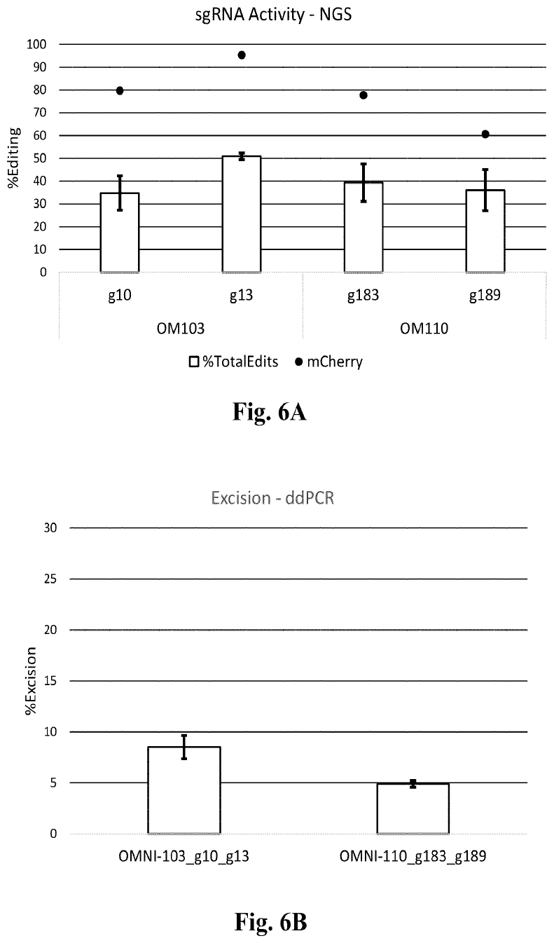

In order to measure the excision levels of the CTG18.1 expanded repeat, excision levels were examined using droplet digital PCR (ddPCR) for two different excising compositions. Briefly, HeLa cells were transfected with the a nuclease and two sgRNAs using JetOptimus. Each sgRNA was also transfected separately in order to assess activity of each guide in this specific experiment. Cells were harvested for next-generation sequencing (NGS) three (3) days after transfection (FIG. 6A), and for genomic extraction and ddPCR 10 days after transfection (FIG. 6B). mCherry was used as a reporter for transfection efficiency and quantified using flow cytometry. Excision was measured using EvaGreen “gain of signal” assay (QX200, BioRad), with primers around the excision area. When no excision occurs, the amplified amplicon is too long to be detected using ddPCR (above 200 bp) and the signal appears only after excision, where the amplified sequence is below 200 bp. The signal was normalized to the RPP30 housekeeping gene. The results showed that for OMNI-103 nuclease, sgRNA10 and sgRNA13 displayed 34.77% and 50.89% editing respectively, and 8.5% excision; for OMNI-110 nuclease, sgRNA183 and sgRNA189 displayed 39.33% and 36.05% editing and 4.89% excision.

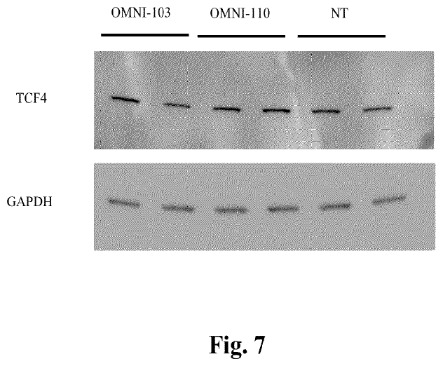

The goal was to perform excision of the expanded repeat without affecting TCF4 expression, since it is a transcription factor that controls central cellular functions. In order to determine whether the excision affects the expression of TCF4, TCF4 protein levels were measured after excision with the indicated compositions, 14 days after transfection. For western blot, cells were lysed with RIPA buffer and 40 μg of total protein were loaded on an SDS page. A specific antibody was used in order to detect protein levels of TCF4 (ab217668, abcam) and GAPDH was used as a loading control. No effect on TCF4 expression was observed (FIG. 7).

### Example 5: Excision Evaluation—Infection with LVLPs in U2OS Cells

Excision using Lentiviral Like Particles (LVLPs) was also tested since this is a feasible delivery system to the corneal endothelial cells. LVLPs are viral particles which contain lentiviral envelope, but they do not contain any viral genetic material—only the sgRNA, which is bound to the structural viral proteins trough aptamer binding proteins (ABP), and the nuclease protein that is recruited by the guide. Upon infection of target cells, the particle can release its RNP content, and transient expression and activity of the released RNP is achieved (Lyu et al., Nucleic Acids Research, 2019).

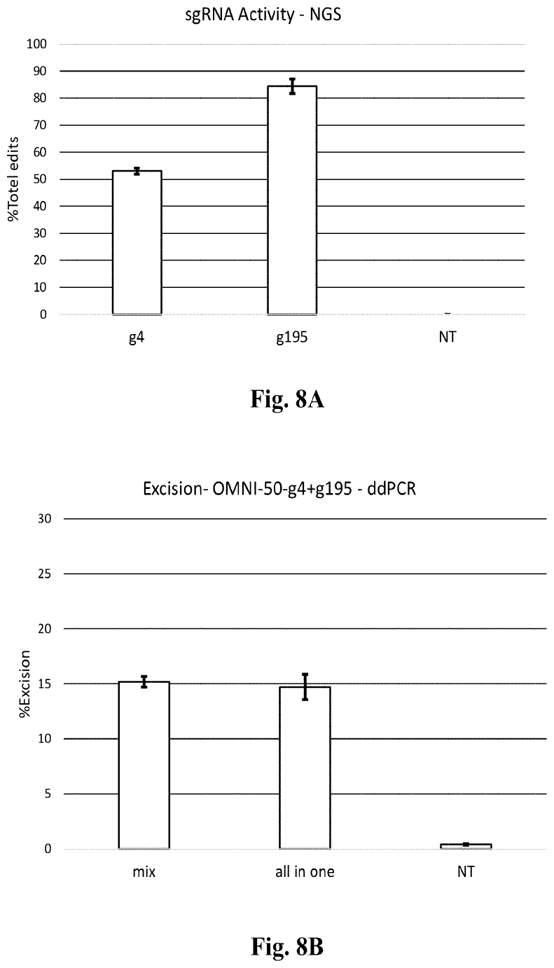

OMNI-50 nuclease is known to be highly active as RNP and it is also established to be efficiently packed in LVLPs. In order to test the activity of OMNI-50, we packed either upstream or downstream RNP composition in LVLPs and tested the editing levels of each sgRNA separately (FIG. 8A) in U2OS cells. sgRNA4 (downstream) and sgRNA195 (upstream) displayed 53.0% and 84.43% editing, respectively, three (3) days after infection (FIG. 8B).

In order to measure excision levels of the expanded repeat area, cells were infected with either the mix of both upstream and downstream LVLP compositions or packed both compositions in the same LVLP product (all in one). In both cases, the excision levels were 15% 18 days after infection (FIG. 8B).

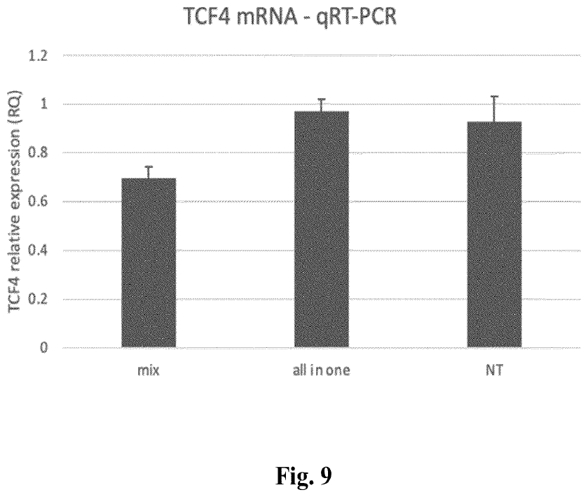

21 days post infection, the above excised cells were subjected to RNA extraction, cDNA was synthesized, and qRT-PCR was performed using fast SYBR and specific primers to the exon11-exon12 junction of TCF4 (FIG. 9). No significant effect was observed on mRNA levels of TCF4 after excision.

### REFERENCES

1. Ahmad and Allen (1992) “Antibody-mediated Specific Binging and Cytotoxicity of Liposome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genome editing without double-strand brakes or donor DNA”. Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjugate Chem 5:382-89.
6. Blaese (1995) “Vectors in cancer therapy: how will they deliver”, Cancer Gene Ther. 2:291-97.
7. Blaese et al. (1995) “T lymphocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1995) “Transfer of genes to humans: early lessons and obstacles to success”, Science 270(5235):404-10.
15. Dillon (1993) “Regulation gene expression in gene therapy” Trends in Biotechnology 11(5):167-173.
16. Dranoff et al. (1997) “A phase I study of vaccination with autologous, irradiated melanoma cells engineered to secrete human granulocyte macrophage colony stimulating factor”, Hum. Gene Ther. 8(1): 111-23.
17. Dunbar et al. (1995) “Retrovirally marked CD34-enriched peripheral blood and bone marrow cells contribute to long-term engraftment after autologous transplantation”, Blood 85:3048-57.
18. Ellem et al. (1997) “A case report: immune responses and clinical course of the first human use of granulocyte/macrophage-colony-stimulating-factor-transduced autologous melanoma cells for immunotherapy”, Cancer Immunol Immunother 44:10-20.
19. Gao and Huang (1995) “Cationic liposome-mediated gene transfer” Gene Ther. 2(10):710-22.
20. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
21. Han et al. (1995) “Ligand-directed retro-viral targeting of human breast cancer cells”, Proc Natl Acad Sci U.S.A. 92(21):9747-51.
22. Hu et al. (2018) “Evolved Cas9 variants with broad PAM compatibility and high DNA specificity”, Nature 556(7699):57-63.
23. Jinek et al. (2012) “A programmable dual-RNA-guided DNA endonuclease in adaptive bacterial immunity,” Science 337(6096):816-21.
24. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
25. Judge et al. (2006) “Design of noninflammatory synthetic siRNA mediating potent gene silencing in vivo”, Mol Ther. 13(3):494-505.
26. Kohn et al. (1995) “Engraftment of gene-modified umbilical cord blood cells in neonates with adenosine deaminase deficiency”, Nature Medicine 1:1017-23.
27. Kremer and Perricaudet (1995) “Adenovirus and adeno-associated virus mediated gene transfer”, Br. Med. Bull. 51(1):31-44.
28. Lyu et al. (2019) “Delivering Cas9/sgRNA ribonucleoprotein (RNP) by lentiviral capsid based bionanoparticles for efficient ‘hit-and-run’ genome editing”, Nucleic Acids Research, 47(17):e99
29. Macdiarmid et al. (2009) “Sequential treatment of drug-resistant tumors with targeted minicells containing siRNA or a cytotoxic drug”, Nat Biotechnol. 27(7):643-51.
30. Malech et al. (1997) “Prolonged production of NADPH oxidase-corrected granulocytes after gene therapy of chronic granulomatous disease”, PNAS 94(22):12133-38.
31. Miller et al. (1991) “Construction and properties of retrovirus packaging cells based on gibbon ape leukemia virus”, J Virol. 65(5):2220-24.
32. Miller (1992) “Human gene therapy comes of age”, Nature 357:455-60.
33. Mitani and Caskey (1993) “Delivering therapeutic genes—matching approach and application”, Trends in Biotechnology 11(5):162-66.
34. Nabel and Felgner (1993) “Direct gene transfer for immunotherapy and immunization”, Trends in Biotechnology 11(5):211-15.
35. Nishimasu et al. (2018) “Engineered CRISPR-Cas9 nuclease with expanded targeting space”, Science 361(6408):1259-1262.
36. Remy et al. (1994) “Gene Transfer with a Series of Lipophilic DNA-Binding Molecules”, Bioconjugate Chem. 5(6):647-54.
37. Sentmanat et al. (2018) “A Survey of Validation Strategies for CRISPR-Cas9 Editing”, Scientific Reports 8:888. doi:10.1038/s41598-018-19441-8.
38. Sommerfelt et al. (1990) “Localization of the receptor gene for type D simian retroviruses on human chromosome 19”, J. Virol. 64(12):6214-20.
39. Van Brunt (1988) “Molecular framing: transgenic animals as bioactors” Biotechnology 6:1149-54.
40. Vigne et al. (1995) “Third-generation adenovectors for gene therapy”, Restorative Neurology and Neuroscience 8(1,2): 35-36.
41. Walton et al. (2020) “Unconstrained genome targeting with near-PAMless engineered CRISPR-Cas9 variants”, Science 368(6488):290-296.
42. Weissman and Kariko (2015) “mRNA:Fulfilling the promise of gene therapy”, Molecular Therapy (9):1416-7.
43. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
44. Yu et al. (1994) “Progress towards gene therapy for HIV infection”, Gene Ther. 1(1):13-26.
45. Zetsche et al. (2015) “Cpf1 is a single RNA-guided endonuclease of a class 2 CRISPR-Cas system” Cell 163(3):759-71.
46. Zuris et al. (2015) “Cationic lipid-mediated delivery of proteins enables efficient protein based genome editing in vitro and in vivo” Nat Biotechnol. 33(1):73-80.

1. A method of excising an intronic trinucleotide CTG repeat expansion from a Transcription Factor 4 (TCF4) allele in a cell, the method comprising
introducing to the cell a composition comprising:
at least one CRISPR nuclease, or a nucleotide molecule encoding a CRISPR nuclease;
a first RNA molecule comprising a first guide sequence portion, or a nucleotide molecule encoding the first RNA molecule; and
a second RNA molecule comprising a second guide sequence portion, or a nucleotide molecule encoding the second RNA molecule,
wherein a complex of the CRISPR nuclease and the first RNA molecule affects a double strand break upstream of the intronic trinucleotide CTG repeat expansion and a complex of the CRISPR nuclease and the second RNA molecule affects a double strand break downstream of the intronic trinucleotide CTG repeat expansion,
wherein the first guide sequence portion comprises 17-50 nucleotides containing at least 17 contiguous nucleotides in the sequence set forth in any one of SEQ ID NOs: 1216-2325 or 46145-47292 and the second guide sequence portion comprises 17-50 nucleotides containing at least 17 contiguous nucleotides in the sequence set forth in any one of SEQ ID NOs: 1-1215 or 45057-45092.

2. The method of claim 1, wherein the first guide sequence portion comprises 17-50 nucleotides containing at least 17 contiguous nucleotides in the sequence set forth in any one of SEQ ID NOs: 1216-2325 and the second guide sequence portion comprises 17-50 nucleotides containing at least 17 contiguous nucleotides in the sequence set forth in any one of SEQ ID NOs: 1-1215 and wherein at least one TCF4 allele in the cell comprises a heterozygous SNP at the rs34071688 position.

3. The method of claim 1, wherein the first guide sequence portion comprises 17-50 nucleotides containing at least 17 contiguous nucleotides in the sequence set forth in any one of SEQ ID NOs: 46145-47292 and the second guide sequence portion comprises 17-50 nucleotides containing at least 17 contiguous nucleotides in the sequence set forth in any one of SEQ ID NOs: 45057-45092 and wherein at least one TCF4 allele in the cell comprises a heterozygous SNP at any one of the positions selected from the group consisting of rs746872826, 18:5558615, rs879522127, and rs1268568114.

4. The method of claim 1, wherein the first guide sequence portion comprises 17-50 nucleotides containing at least 17 contiguous nucleotides in the sequence set forth in any one of SEQ ID NOs: 1216-2325 and the second guide sequence portion comprises 17-50 nucleotides containing at least 17 contiguous nucleotides in the sequence set forth in any one of SEQ ID NOs: 1-1215 and wherein at least one TCF4 allele in the cell comprises a heterozygous SNP at any one of the SNP positions selected from the group consisting of rs71670792, rs1261085, rs1261084, rs11431395, rs373174214, rs35555522, rs66807288, rs141970461, rs71674214, rs8766, rs10221362, rs2276195, rs569385112, rs398041379, rs72925008, rs8090085, rs5825130, rs56887277, rs397836157, rs55755941, rs781691419, rs11409023, rs10221357, rs11661961, rs11660565, rs61656112, rs60565673, rs72925018, rs1261073, rs1261076, rs748718974, rs13381800, rs34773632, rs1942265, rs7241077, rs62092440, rs1893431, rs3838898, rs1893430, 18:55251930_G_GTTTT, rs771385941, rs4800988, rs1942264, rs1261093, rs1539950, rs1539951, rs62092442, rs62092444, rs11662842, rs11664992, rs1261134, rs1261114, rs1788027, rs397858367, rs113662542, rs1261118, rs10701336, rs397809469, rs150323043, rs1038226655, rs149454001, rs796169498, rs9955026, rs1046741326, rs754435392, rs867471715, rs777608256, rs762153709, rs1153636, rs1153637, rs5825134, rs35480166, rs893946, rs374155330, rs781053385, rs899293868, rs996440450, rs1349129287, rs780424692, rs34702622, rs11428164, rs1660235, rs1440473, rs1788026, rs11332509, rs1660237, rs1631486, rs1025804, rs753933037, rs1660233, rs200650987, rs71951255, rs368762262, rs751007744, rs780342991, rs1660241, rs796749696, rs61468075, rs777518462, rs1660242, rs1440476, rs1011392, 18:55374871_T_TA, rs1788030, rs1623427, rs1621581, rs1788025, rs1788023, rs1348047, rs1788019, rs9950000, rs9958125, rs9320010, rs3794891, rs757629087, rs772409228, rs12607679, rs3794889, rs4801149, rs12605773, rs2872041, rs4801150, rs1020169, rs7238888, rs7235757, rs2958178, rs2958165, rs2958171, rs1328839434, rs796792902, rs2958175, rs11309751, rs2958186, rs2919446, rs2958161, rs2860511, rs2958162, rs2919451, rs2919450, rs201657057, rs2958163, rs1440477, rs2958166, rs377458803, rs2958169, rs8098843, rs11412305, rs386387765, 18:55423173_T_TA, rs781071274, rs4374254, rs370693034, rs761981780, rs8084308, rs77540208, rs9320016, rs4524013, rs1025639279, rs4500831, rs7229456, rs12967143, rs12963334, rs12963463, rs398100891, rs12958048, 18:55434419_C_CTTT, rs375388593, rs140134419, rs4801153, rs4801154, rs745460290, rs527450659, rs4341827, rs4468713, rs7228159, rs145330990, rs7231748, rs34577882, rs34578042, rs1452789, rs1452788, rs12606995, rs188225813, rs732779, rs11385247, rs9966430, rs2924321, rs151196106, rs3760600, rs2924328, rs1377243, rs2924329, rs5825142, rs199707137, 18:55468891_C_CCCA, rs11338618, rs149728054, rs11298284, rs7233312, rs2924331, 18:55480276_C_CAA, rs2958182, rs2958183, rs2958184, rs2924332, rs2924333, rs2060889, rs2958187, rs2924335, rs138885827, rs2924336, rs59413482, rs796565215, 18:55496896_C_CAA, rs4801157, rs2958188, rs2958189, rs2060886, rs3017183, rs2958158, rs2924338, rs12956276, rs55812411, rs776881842, rs1452791, rs9957668, rs9954890, rs9964328, rs67387556, rs1491335073, 18:55511330_T_TAAA, rs751932079, rs2957261, 18:55511331_T_TAAAA, rs8090106, rs140221855, rs17089851, rs398032944, rs1341922999, rs624244, rs627685, rs9948513, rs9965067, rs9965195, rs35371867, rs11441646, rs9949107, rs7240986, rs4801158, rs72627231, rs11412432, rs33938531, rs4800990, rs4458089, rs4572488, rs12968271, rs9636107, rs2123389, rs9947814, rs71352207, rs1452787, rs2123392, rs2123393, 18:55559041_T_TA, rs34935191, rs74182105, rs139870092, rs76053687, rs150848781, rs564960433, rs41396445, and rs34232463.

5. (canceled)

6. The method of claim 1, wherein the cell is a corneal cell or a corneal endothelial cell.

7. (canceled)

8. The method of claim 1, wherein the composition is introduced to the cell by a lentivirus-like particle (LVLP).

9. The method of claim 1, wherein the cell is a stem cell, a fibroblast, blood cell, hepatocyte, keratinocyte, any other cell type capable of being reprogrammed to an induced pluripotent stem cell (iPSC), a hematopoietic stem cell (HSC), an induced pluripotent stem cell (iPS cell), an iPSc-derived cell, or an iPSc-derived corneal endothelial cell.

10. (canceled)

11. (canceled)

12. (canceled)

13. (canceled)

14. A composition comprising an RNA molecule having a guide sequence portion that comprises 17-50 nucleotides containing at least 17 contiguous nucleotides in the sequence set forth in any one of SEQ ID NOs: 1-2325, 45057-45092, or 46145-47292.

15. The composition of claim 14, further comprising a second RNA molecule, wherein the first RNA molecule has a guide sequence portion that comprises 17-50 nucleotides containing at least 17 contiguous nucleotides in the sequence set forth in any one of SEQ ID NOs: 1216-2325, and the second RNA molecule has a guide sequence portion that comprises 17-50 nucleotides containing at least 17 contiguous nucleotides in the sequence set forth in any one of SEQ ID NOs: 1-1215.

16. The composition of claim 14, further comprising a CRISPR nuclease.

17. (canceled)

18. A cell modified by the composition of claim 14.

19. The modified cell of claim 18, wherein the cell is a corneal cell or corneal endothelial cell.

20. The modified cell of claim 18, wherein the cell is a stem cell, or a fibroblast, blood cell, hepatocyte, keratinocyte, or any other cell type capable of being reprogrammed to an induced pluripotent stem cell (iPSC), a hematopoietic stem cell (HSC), iPSC-derived cell, or an iPSC-derived corneal endothelial cell.

21. (canceled)

22. (canceled)

23. A method of treating Fuchs Endothelial Corneal Dystrophy (FECD) in a human subject, the method comprising delivering the composition of claim 14 to the subject.

24. (canceled)

25. The method of claim 23, wherein the composition is introduced to the cell by a lentivirus-like particle (LVLP).

26. (canceled)

27. (canceled)

28. Use of the modified cell of claim 18 for treating FECD, comprising delivering the modified cell to a subject having or at risk of having FECD.

29. (canceled)

30. A kit for excising an intronic trinucleotide CTG repeat expansion from a TCF4 allele in a cell, comprising the composition of claim 14 and instructions for delivering the composition to the cell.

31. (canceled)

32. (canceled)

33. (canceled)

34. (canceled)

35. The composition of claim 14, wherein the guide sequence portion of the RNA molecule comprises 17-50 nucleotides containing at least 17 contiguous nucleotides in the sequence set forth in any one of SEQ ID NOs: 178, 238, 308, 405, 830, 907, 973, 1007, 1040, 1053, 1078, 1138, 1141, 1173, 1214-1216, 1436, 1541, 1575, 1940, 1986, 1970, 2007, 2026, 2036, 2039, 2041, 2103, 2144, 2151, 2167, 2170, 2180, 2200, 2212, 2285, or 2319-2325.

36. The composition of claim 15,
i) wherein the guide sequence portion of the first RNA molecule comprises 17-50 nucleotides containing at least 17 contiguous nucleotides in the sequence set forth in SEQ ID: 1216 and the guide sequence portion of the second RNA molecule comprises 17-50 nucleotides containing at least 17 contiguous nucleotides in the sequence set forth in SEQ ID: 238;
ii) wherein the guide sequence portion of the first RNA molecule comprises 17-50 nucleotides containing at least 17 contiguous nucleotides in the sequence set forth in SEQ ID: 1436 and the guide sequence portion of the second RNA molecule comprises 17-50 nucleotides containing at least 17 contiguous nucleotides in the sequence set forth in SEQ ID: 178, 238, or 308;
iii) wherein the guide sequence portion of the first RNA molecule comprises 17-50 nucleotides containing at least 17 contiguous nucleotides in the sequence set forth in SEQ ID: 1541 and the guide sequence portion of the second RNA molecule comprises 17-50 nucleotides containing at least 17 contiguous nucleotides in the sequence set forth in SEQ ID: 178 or 308;
iv) wherein the guide sequence portion of the first RNA molecule comprises 17-50 nucleotides containing at least 17 contiguous nucleotides in the sequence set forth in SEQ ID: 1575 and the guide sequence portion of the second RNA molecule comprises 17-50 nucleotides containing at least 17 contiguous nucleotides in the sequence set forth in SEQ ID: 178, 238, 308, or 405;
v) wherein the guide sequence portion of the first RNA molecule comprises 17-50 nucleotides containing at least 17 contiguous nucleotides in the sequence set forth in SEQ ID: 1970 and the guide sequence portion of the second RNA molecule comprises 17-50 nucleotides containing at least 17 contiguous nucleotides in the sequence set forth in SEQ ID: 1138;
vi) wherein the guide sequence portion of the first RNA molecule comprises 17-50 nucleotides containing at least 17 contiguous nucleotides in the sequence set forth in SEQ ID: 2170 and the guide sequence portion of the second RNA molecule comprises 17-50 nucleotides containing at least 17 contiguous nucleotides in the sequence set forth in SEQ ID: 1053; or
vii) wherein the guide sequence portion of the first RNA molecule comprises 17-50 nucleotides containing at least 17 contiguous nucleotides in the sequence set forth in SEQ ID: 2200 and the guide sequence portion of the second RNA molecule comprises 17-50 nucleotides containing at least 17 contiguous nucleotides in the sequence set forth in SEQ ID: 1078.

37. The composition of claim 36, wherein the composition excises an intronic trinucleotide CTG repeat expansion from a TCF4 allele in a cell without affecting the expression of TCF4.
